(19) United States
(12) Patent Application Publication
(10) Pub. No.: US 2014/0271717 A1
(43) Pub. Date: Sep. 18, 2014

(54) DEVICES CONTAINING A CHEMICAL DENERVATION AGENT AND METHODS FOR TREATING CHRONIC BACK PAIN USING CHEMICAL DENERVATION

(71) Applicant: KYPHON SARL, NEUCHATEL (CH)

(72) Inventors: MOJAN GOSHAYESHGAR, Atherton, CA (US); Michael A. Smith, San Jose, CA (US)

(73) Assignee: KYPHON SARL, NEUCHATEL (CH)

(21) Appl. No.: 13/803,296

(22) Filed: Mar. 14, 2013

(51) Int. Cl. A61K 9/00

(52) U.S. Cl. CPC A61K 9/0085
USPC 424/239.1; 424/94.62; 514/724; 424/680

(57) ABSTRACT

Effective devices and methods using a chemical denervation agent are provided for treating chronic back pain. The devices and methods comprise a chemical denervation agent to degrade or to shrink at least a portion of a nerve associated with back pain e.g. basivertebral nerve of the lumbar. In some embodiments, the methods and devices are configured to immediately release an effective amount of the chemical denervation agent within 24 hours and provide sustained release of the chemical denervation agent or other therapeutic agent over a period of up to one year to treat chronic back pain.

### BACKGROUND

The vertebra maybe damaged due to trauma or disease. Damage of the vertebra may cause end plates of the vertebrae to collapse and cause pressure on nerves in the vertebrae resulting in chronic back pain. Thus, destroying or interrupting the nerve will result in reduced back pain.

Multiple studies indicated that the basivertebral nerve conducts pain receptive signals from vertebral endplates adjacent to degenerated disks. This results from the compression or collapse of the vertebral endplates, leading to the compression of the basivertebral nerves. Chemical denervation of the lumbar basivertebral nerve may provide relief to patients with chronic lower back pain. Thus, there is a need to develop new devices and methods of treating chronic back pain caused by the degeneration, of vertebral, namely the lumbar region of the spine that allow accurate and precise delivery of chemical denervation agents at, near, or in the damaged area of the vertebra resulting in minimal physical and psychological trauma to the patient and reductions in chronic back pain.

### SUMMARY

New devices and methods are provided for the treatment of chronic back pain that allow accurate and precise delivery of chemical denervation agents at, near, or in the damaged area of the vertebra resulting in minimal physical and psychological trauma to the patient and reductions in chronic back pain.

In some embodiments, the chemical denervation agent can be administered in the same cannula or needle without the need to reposition it several times. The chemical denervation agent ablates the basivertebral nerve to permanently block neural transmission in the treatment zone to reduce chronic back pain in the patient.

In one embodiment, a device for treating chronic back pain in a patient in need of such treatment is provided. The device is configured to deliver a chemical denervation agent to an effective treatment zone comprising a nerve at a target region of the spine so as to chemically ablate the nerve.

In another embodiment, a device for treating chronic back pain in a patient in need of such treatment is provided. The device being implantable in or near the basivertebral nerve of the patient. The device comprising a chemical denervation agent and having an immediate release component configured to release an effective amount of the chemical denervation agent within 24 hours to ablate at least a portion of the vertebral nerve e.g. basivertebral nerve, of the patient.

In yet another embodiment, there is a method for treating chronic back pain in a patient in need of such treatment, the method comprising the steps of positioning a delivery device for delivery of a chemical denervation agent in or near an effective treatment zone containing a basivertebral nerve for administering a chemical denervation agent to chemically ablate at least a portion of the basivertebral nerve so as to treat chronic back pain of the patient.

Additional features and advantages of various embodiments will be set forth in part in the description that follows, and in part will be apparent from the description, or may be learned by practice of various embodiments. The objectives and other advantages of various embodiments will be realized and attained by means of the elements and combinations particularly pointed out in the description and appended claims.

### BRIEF DESCRIPTION OF THE DRAWINGS

In part, other aspects, features, benefits and advantages of the embodiments will be apparent with regard to the following description, appended claims and accompanying drawings where:

FIG. 1 illustrates a sagittal view of a section of a vertebral column showing vertebral intraosseous innervation of the basivertebral nerve.

FIG. 2 illustrates an embodiment of an vertebrae treatment including inserting a cannula or needle to deliver a chemical denervation agent to an effective treatment zone comprising a nerve at a target region of the spine.

FIG. 3 illustrates an embodiment of an vertebrae treatment including administering a chemical denervation agent into the effective treatment zone of the vertebrae including the basivertebral nerve.

FIG. 4 illustrates an embodiment of a vertebrae treatment including delivering an implantable drug depot, comprising a chemical denervation agent, into the effective treatment zone of the vertebrae.

FIG. 5 illustrates an enlarged view of a drug depot containing an immediate release layer that immediately releases a chemical denervation agent, a sustained release layer that releases the chemical denervation agent over a prolonged period of time, and section that may contain additional agents.

FIG. 6 illustrates an enlarged view of an embodiment of a drug depot containing an immediate release portion that immediately releases a chemical denervation agent and/or other therapeutic agent and a sustained release portion in microspheres that releases the chemical denervation agent and/or other therapeutic agent over a prolonged period of time.

It is to be understood that the figures are not drawn to scale. Further, the relation between objects in a figure may not be to scale, and may in fact have a reverse relationship as to size. The figures are intended to bring understanding and clarity to the structure of each object shown, and thus, some features may be exaggerated in order to illustrate a specific feature of a structure.

### DETAILED DESCRIPTION

For the purposes of this specification and appended claims, unless otherwise indicated, all numbers expressing quantities of ingredients, percentages or proportions of materials, reaction conditions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding the numerical ranges and parameters set forth herein, the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “1 to 10” includes any and all subranges between (and including) the minimum value of 1 and the maximum value of 10, that is, any and all subranges having a minimum value of equal to or greater than 1 and a maximum value of equal to or less than 10, e.g., 5.5 to 10.

Reference will now be made in detail to certain embodiments of the invention, examples of which are illustrated in the accompanying drawings. While the invention will be described in conjunction with the illustrated embodiments, it will be understood that they are not intended to limit the invention to those embodiments. On the contrary, the invention is intended to cover all alternatives, modifications, and equivalents that may be included within the invention as defined by the appended claims.

The headings below are not meant to limit the disclosure in any way; embodiments under any one heading may be used in conjunction with embodiments under any other heading.

### DEFINITIONS

It is noted that, as used in this specification and the appended claims, the singular forms “a,” “an,” and “the,” include plural referents unless expressly and unequivocally limited to one referent. Thus, for example, reference to “a drug depot” includes one, two, three or more drug depots.

An “implantable device” and expressions of like as utilized herein refers to any object implantable through surgical, medical, injection, or other suitable means whose primary function is achieved either through its physical presence or mechanical properties. Implantable devices include one or more drug depots.

A “chemical denervation agent”, includes, but is not limited to an agent that permanently blocks neural transmission to reduce chronic pain in the patient, such as for example, the ablation of the basivertebral nerve in the treatment zone of the intervertebral body to reduce chronic back pain. For example, in some embodiments the chemical denervation agent can be a neurolytic agent such as Ethanol or a neurotoxin agent such as Botulinum Toxin. Localized drug delivery may also include products including magnetic nano particles that contain neurotoxins like for example Botox B.

“Analgesic” refers to an agent or compound that can reduce, relieve or eliminate pain. Examples of analgesic agents include but are not limited to acetaminophen, a local anesthetic, such as for example, lidocaine, bupivacaine, ropivacaine, opioid analgesics such as buprenorphine, butorphanol, dextromoramide, dezocine, dextropropoxyphene, diamorphine, fentanyl, alfentanil, sufentanil, hydrocodone, hydromorphone, ketobemidone, levomethadyl, levorphanol, mepiridine, methadone, morphine, nalbuphine, opium, oxycodone, papavereturn, pentazocine, pethidine, phenoperidine, piritramide, dextropropoxyphene, remifentanil, sufentanil, tilidine, tramadol, codeine, dihydrocodeine, meptazinol, dezocine, eptazocine, flupirtine or a combination thereof. Analgesic agents also include those with analgesic and anti-inflammatory properties, such as, for example, amitriptyline, carbamazepine, gabapentin, pregabalin, clonidine, or a combination thereof. The device can include one or more analgesics.

The phrase “anti-inflammatory agent” refers to an agent or compound that has anti-inflammatory effects. The device can include one or more anti-inflammatory agents. These agents may remedy pain by reducing inflammation. Examples of anti-inflammatory agents include, but are not limited to, a statin, sulindac, sulfasalazine, naroxyn, diclofenac, indomethacin, ibuprofen, flurbiprofen, ketoprofen, aclofenac, aloxiprin, aproxen, aspirin, diflunisal, fenoprofen, mefenamic acid, naproxen, phenylbutazone, piroxicam, meloxicam, salicylamide, salicylic acid, desoxysulindac, tenoxicam, ketoralac, clonidine, flufenisal, salsalate, triethanolamine salicylate, aminopyrine, antipyrine, oxyphenbutazone, apazone, cintazone, flufenamic acid, clonixeril, clonixin, meclofenamic acid, flunixin, colchicine, demecolcine, allopurinol, oxypurinol, benzydamine hydrochloride, dimefadane, indoxole, intrazole, mimbane hydrochloride, paranylene hydrochloride, tetrydamine, benzindopyrine hydrochloride, fluprofen, ibufenac, naproxol, fenbufen, cinchophen, diflumidone sodium, fenamole, flutiazin, metazamide, letimide hydrochloride, nexeridine hydrochloride, octazamide, molinazole, neocinchophen, nimazole, proxazole citrate, tesicam, tesimide, tolmetin, triflumidate, fenamates (mefenamic acid, meclofenamic acid), nabumetone, celecoxib, etodolac, nimesulide, apazone, gold, tepoxalin; dithiocarbamate, or a combination thereof. Anti-inflammatory agents also include other compounds such as steroids, such as for example, fluocinolone, cortisol, cortisone, hydrocortisone, fludrocortisone, prednisone, prednisolone, methylprednisolone, triamcinolone, betamethasone, dexamethasone, beclomethasone, fluticasone interleukin-1 receptor antagonists, thalidomide (a TNF-α release inhibitor), thalidomide analogues (which reduce TNF-α production by macrophages), bone morphogenetic protein (BMP) type 2 or BMP-4 (inhibitors of caspase 8, a TNF-α activator), quinapril (an inhibitor of angiotensin II, which upregulates TNF-α), interferons such as IL-11 (which modulate TNF-α receptor expression), and aurin-tricarboxylic acid (which inhibits TNF-α), guanidinoethyldisulfide, or a combination thereof.

Exemplary anti-inflammatory agents include, for example, naproxen; diclofenac; celecoxib; sulindac; diflunisal; piroxicam; indomethacin; etodolac; meloxicam; ibuprofen; ketoprofen; r-flurbiprofen; mefenamic; nabumetone; tolmetin, and sodium salts of each of the foregoing; ketorolac bromethamine; ketorolac tromethamine; ketorolac acid; choline magnesium trisalicylate; rofecoxib; valdecoxib; lumiracoxib; etoricoxib; aspirin; salicylic acid and its sodium salt; salicylate esters of alpha, beta, gamma-tocopherols and tocotrienols (and all their d, 1, and racemic isomers); methyl, ethyl, propyl, isopropyl, n-butyl, sec-butyl, t-butyl, esters of acetylsalicylic acid; tenoxicam; aceclofenac; nimesulide; nepafenac; amfenac; bromfenac; flufenamate; phenylbutazone, or a combination thereof.

The device can include one or more steroids. Exemplary steroids include, for example, 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, dexamethasone 21-acetate, dexamethasone 21-phosphate di-Na salt, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide or a combination thereof.

The device can include one or more statins. Examples of useful statins for treatment of pain and/or inflammation include, but are not limited to, atorvastatin, simvastatin, pravastatin, cerivastatin, mevastatin (see U.S. Pat. No. 3,883,140, the entire disclosure is herein incorporated by reference), velostatin (also called synvinolin; see U.S. Pat. Nos. 4,448,784 and 4,450,171 these entire disclosures are herein incorporated by reference), fluvastatin, lovastatin, rosuvastatin and fluindostatin (Sandoz XU-62-320), dalvastain (EP Appln. Publn. No. 738510 A2, the entire disclosure is herein incorporated by reference), eptastatin, pitavastatin, or pharmaceutically acceptable salts thereof or a combination thereof. In various embodiments, the statin may comprise mixtures of (+)R and (−)—S enantiomers of the statin. In various embodiments, the statin may comprise a 1:1 racemic mixture of the statin. Anti-inflammatory agents also include those with anti-inflammatory properties, such as, for example, amitriptyline, carbamazepine, gabapentin, pregabalin, clonidine, or a combination thereof.

In some embodiments, the anti-inflammatory agent can include an “anti-cytokine agent.” An anti-cytokine agent includes any molecule, cell, or physical stimulus which decreases, blocks, inhibits, abrogates or interferes with the pro-inflammatory cascade of cytokine proteins leading to an inflammatory response. For example, a suitable “tumor necrosis factor alpha antagonist” or “TNF-alpha” antagonist can bind TNF, and includes anti-TNF antibodies and/or receptor molecules which bind specifically to TNF. A suitable TNF antagonist can also prevent or inhibit TNF synthesis and/or TNF release and includes compounds such as thalidomide, tenidap, or phosphodiesterase inhibitors, such as, but not limited to, pentoxifylline or rolipram.

Anti-cytokine agents include substances that are direct and local-acting modulators of the pro-inflammatory effect of TNF-alpha, such as but not limited to, soluble tumor necrosis factor alpha receptors, any pegylated soluble tumor necrosis factor alpha receptor, monoclonal or polyclonal antibodies or antibody fragments or combinations thereof. Suitable examples include but are not limited to Adalimumab, Infliximab, Etanercept, Pegsunercept (PEG sTNF-R1), sTNF-R1, CDP-870, CDP-571, CNI-1493, RDP58, ISIS 104838, 1, 3-beta-D-glucans, Lenercept, PEG-sTNFRII Fc Mutein, D2E7, Afelimomab, or combinations thereof. They can decrease pain through their actions as inhibitors or agonists of the release of pro-inflammatory molecules. For example, these substances can act by inhibiting or antagonizing expression or binding of cytokines or other molecules that act in the early inflammatory cascade, often resulting in the downstream release of prostaglandins and leukotrienes. These substances can also act, for example, by blocking or antagonizing the binding of excitatory molecules to nociceptive receptors in the nervous system or neuromuscular system, as these receptors often trigger an inflammatory response to inflammation or injury of the nerve or surrounding tissue through a nitric oxide-mediated mechanism. These biological response modifiers include, for example, inhibitors of the action of tumor necrosis factor alpha (TNF-alpha).

In one example of an alternative approach, the anti-cytokine agent is a TNF binding protein. One suitable such anti-cytokine agent is currently referred to as Onercept, Onercept-like agents, and derivatives are all considered acceptable. Still other suitable anti-cytokine agents include dominant-negative TNF variants. A suitable dominant-negative TNF variant includes but is not limited to DN-TNF and including those described by Steed et al. (2003), “Inactivation of TNF signaling by rationally designed dominant-negative TNF variants,” Science, 301 (5641):1895-1898. Still more embodiments include the use of a recombinant adeno-associated viral (rAAV) vector technology platform to deliver the oligonucleotides encoding inhibitors, enhancers, potentiators, neutralizers, or other modifiers. For example, in one embodiment a rAAV vector technology platform delivers the DNA sequence of a potent inhibitor of tumor necrosis factor (TNF-alpha). One suitable inhibitor is TNFR:Fc. Other anti-cytokine agents interfere with one of the steps in the gene expression and secretion of cytokines, such as transcription, translation, folding, post-translational modification, and intracellular transport. For example, small anti-sense RNA or short interfering RNA (siRNA) can block post-transcriptional processing of cytokine genes. Other anti-cytokine agents include antibodies, including but not limited to naturally occurring or synthetic, double chain, single chained, or fragments thereof. For example, suitable anti-cytokine agents include molecules are based on single chain antibodies called Nanobodies® (Ablynx, Ghent Belgium) which are defined as the smallest functional fragment of a naturally-occurring single domain antibody.

It is understood that TNF is both affected by upstream events which modulate its production and, in turn, affects downstream events. Alternative approaches to treating chronic back pain include using antagonists designed to specifically target TNF as well as molecules upstream, downstream and/or a combination thereof. Such approaches include, but are not limited to modulating TNF directly, modulating kinases, inhibiting cell-signaling, manipulating second messenger systems, modulating kinase activation signals, modulating a cluster designator on an inflammatory cell, modulating other receptors on inflammatory cells, blocking transcription or translation of TNF or other targets in pathway, modulating TNF-alpha post-translational effects, employing gene silencing, or modulating interleukins, for example IL-1, IL-6 and IL-8.

Interleukin-1 is a pro-inflammatory cytokine similar in action to TNF-alpha. For example, certain inhibitors of this protein are similar to those developed to inhibit TNF-alpha. One such example is Kineret® (anakinra) which is a recombinant, non-glycosylated form of the human interleukin-1 receptor antagonist (IL-1Ra). Another suitable anti-cytokine agent is AMG 108, which is a monoclonal antibody that blocks the action of IL-1.

Other suitable anti-cytokine agents include: integrin antagonists, alpha-4 beta-7 integrin antagonists, cell adhesion inhibitors, interferon gamma antagonists, CTLA4-Ig agonists/antagonists (BMS-188667), CD40 ligand antagonists, Humanized anti-IL-6 mAb (MRA, Tocilizumab, Chugai), HMGB-1 mAb (Critical Therapeutics Inc.), anti-IL2R antibody (daclizumab, basilicimab), ABX (anti IL-8 antibody), recombinant human IL-10, and HuMax IL-15 (anti-IL 15 antibody).

Unless otherwise specified or apparent from context, where this specification and the set of claims that follows refer to a drug (e.g., a chemical denervation agent, an anti-inflammatory agent, analgesic, or the like) the inventor(s) are also referring to a pharmaceutically acceptable salt of the drug including stereoisomers. Pharmaceutically acceptable salts include those salt-forming acids and bases that do not substantially increase the toxicity of the compound. Some examples of potentially suitable salts include salts of alkali metals such as magnesium, calcium, sodium, potassium and ammonium, salts of mineral acids such as hydrochloric, hydriodic, hydrobromic, phosphoric, metaphosphoric, nitric and sulfuric acids, as well as salts of organic acids such as tartaric, acetic, citric, malic, benzoic, glycollic, gluconic, gulonic, succinic, arylsulfonic, e.g., p-toluenesulfonic acids, or the like.

“Treating” or treatment of a disease or condition refers to executing a protocol, which may include administering one or more drugs, such as a chemical denervation agent, to a patient (human, normal or otherwise, or other mammal), in an effort to alleviate signs or symptoms of the disease. Alleviation can occur prior to signs or symptoms of the disease or condition appearing, as well as after their appearance. Thus, “treating” or “treatment” includes “preventing” or “prevention” of disease or undesirable condition. In addition, “treating” or “treatment” does not require complete alleviation of signs or symptoms, does not require a cure, and specifically includes protocols that have only a marginal effect on the patient. “Reducing pain” includes a decrease in pain and does not require complete alleviation of pain signs or symptoms, and does not require a cure. In various embodiments, reducing pain includes even a marginal decrease in pain. By way of example, the administration of a medical device (e.g., drug depot) containing a chemical denervation agent degrades the basivertebral nerve in the lumber vertebrae of a patient, which reduces or alleviates chronic back pain, in some embodiments, additional agents administered with the chemical denervation agent, reduces pain and/or inflammation.

“Localized” delivery includes delivery where one or more devices (e.g., drug depots) containing at least the chemical denervation agent is deposited within a tissue, for example, a lumbar vertebrae, in close proximity (within about 5 cm, or preferably within about 2 cm, for example, to a nerve such as the basivertebral nerve. In an embodiment, alternative a catheter can be used to deliver a chemical denervation agent t an area containing a nerve to be ablated. A “targeted delivery system” provides delivery of one or more chemical denervation agents in either one or more drugs depots having a quantity of chemical denervation agents and/or therapeutic agent that can be deposited at or near the target site as needed for nerve ablation and/or treatment of pain, inflammation or other disease or condition.

The term “mammal” refers to organisms from the taxonomy class “mammalian,” including but not limited to humans, other primates such as chimpanzees, apes, orangutans and monkeys, rats, mice, cats, dogs, cows, horses, etc. In various embodiments, the mammal is a human patient.

### Chemical Denervation Agent

New compositions and methods of chronic back pain are provided that allow accurate and precise implantation of a device (e.g., drug depot catheter to deliver agents) comprising a chemical denervation agent at, near, or in the nerve to be ablated resulting in minimal physical and psychological trauma to the patient.

By the administration of a medical device having the chemical denervation agent disposed therein, accurate and precise implantation of the chemical denervation agent at, near, or in the nerve to be ablated resulting in minimal physical and psychological trauma to the patient can be accomplished. In some embodiments, the chemical denervation agent can be administered in the same catheter or needle without the need to reposition it several times. The chemical denervation agent ablates the nerve, for example, the basivertebral nerve, to permanently block neural transmission of the treatment zone to reduce chronic back pain in the patient.

In one embodiment, a device is provided for treating chronic back pain due to vertebrae destruction in a patient in need of such treatment, the device being biodegradable and implantable within the vertebrae, the device comprising a chemical denervation agent to ablate or proteolytically degrade at least a portion of the basivertebral nerve and being configured to immediately release an effective amount of the chemical denervation agent within 24 hours and provide sustained release of the chemical denervation agent or other therapeutic agent over a period of up to one year to ablate the basivertebral nerve over time and reduce chronic back pain.

The device comprises one or more chemical denervation agents, which degrade or cause the dissolution of the basivertebral nerve or a portion thereof, which reduce or permanently blocks neural transmission of the nerve in the treatment zone reducing chronic back pain and/or inflammation associated therewith.

Chemical denervation agents include, one or more neurolytic agents such as Ethanol or a neurotoxin agent such as Botulinum Toxin. Localized drug delivery may also include products including magnetic nano particles that contain neurotoxins like for example Botox B.

In some embodiments of the methods provided herein, the chemical denervation agent is administered in an amount sufficient to maintain a pharmacologically active level of the chemical denervation agent locally at the site of implantation in an amount to degrade at least a portion of the basivertebral nerve of the lumbar vertebrae which reduces or permanently blocks neural transmission of the nerve. This will reduce pain and/or inflammation at the site. For example, at least one or more chemical neurological agent is used to ablate the nerve at each level. In some embodiments, the amount of chemical denervation agent released from the device is released as an initial burst and then over time.

In some embodiments of the methods provided herein, the chemical denervation agent is hyaluronidase. Hyaluronidase is available from various manufactures and is described in U.S. Pat. Nos. 7,767,429; 7,169,405; 7,132,098; 7,572,440; 6,958,149; and U.S. Publication Nos. US20040268425; US20100003238; US20090214505; US20100003237; and WO/2009/111066. The entire disclosures of these patents and publications are herein incorporated by reference in their entirety into the present disclosure. One form of hyaluronidase suitable for use in the device is available from Halozyme Therapeutics, Inc. (IL USA), which is a recombinant human hyaluronidase glycoprotein enzyme platform (rHuPH20). The hyaluronidase can be pegylated or a pegylated variant and incorporated into the device (e.g., drug depot).

In some embodiments, the chemical denervation agent and optionally one or more additional therapeutic agents (e.g., growth factor, analgesic, anti-inflammatory agent, etc.) are included in a device that is a drug depot. A “drug depot” comprises the composition in which at least one therapeutic agent or active pharmaceutical ingredient or drug is administered to the body. Thus, a drug depot may comprise a physical structure to facilitate implantation and retention in a desired site (e.g., a vertebrae, a spinal canal, a tissue of the patient, or site of pain and/or inflammation, etc.). The drug depot also comprises the drug itself. The term “drug” as used herein is generally meant to refer to any substance that alters the physiology of a patient. The term “drug” may be used interchangeably herein with the terms “therapeutic agent,” “therapeutically effective amount,” and “active pharmaceutical ingredient” or “API.” It will be understood that unless otherwise specified a “drug” formulation may include more than one therapeutic agent, wherein exemplary combinations of therapeutic agents include a combination of two or more drugs. The drug provides a concentration gradient of a chemical denervation agent as well as an anti-inflammatory agent for delivery to the site. In various embodiments, the drug depot provides an optimal drug concentration gradient of the therapeutic agent at a distance of up to about 0.1 cm to about 5 cm from the implant site, and comprises at least one therapeutic agent or its pharmaceutically acceptable salt.

The term “therapeutic agent” includes any molecule, protein, growth factor, etc. which would be contemplated for administration in, at or near the basivertebral nerve in a vertebrae. This would be delivered in addition to the chemical denervation agent. Such examples would include, but are not limited to one or more growth factors, anti-inflammatory agents (e.g., NSAIDS), antibiotics, analgesics, muscle relaxants, or the like, as well as any molecule or cell, which decreases, blocks, inhibits, abrogates or interferes with the pro-inflammatory cascade of proteins leading to an inflammatory response. For example, a suitable TNF-α antagonist can bind TNF-α, and includes anti-TNF-α antibodies and/or receptor molecules which bind specifically to TNF-α, as well as small molecules which antagonize TNF-α activity. A suitable TNF-α antagonist can also prevent or inhibit TNF-α synthesis and/or TNF-α release. Another example may also provide for any cytokine or biologically active fragment thereof which possesses the ability to decrease, block, inhibit, abrogate or interfere with the pro-inflammatory response promoted by other cytokine proteins (e.g., IL-10, IL-4, IL-13 and TGF-β) as well as any molecule, cell, which positively modulates the anti-inflammatory effect of such an anti-inflammatory cytokine so as to impart an increase in the ability to reduce patient inflammation and/or pain.

The therapeutic agent may comprise growth factors that modulate the growth or differentiation of other cells, particularly connective tissue progenitor cells. The therapeutic agent may include, but is not limited to, members of the fibroblast growth factor family, including acidic and basic fibroblast growth factor (FGF-1 and FGF-2) and FGF-4, members of the platelet-derived growth factor (PDGF) family, including PDGF-AB, PDGF-BB and PDGF-AA; EGFs; the TGF-β superfamily, including TGF-β1, 2 or 3; osteoid-inducing factor (OIF); angiogenin(s); endothelins; hepatocyte growth factor or keratinocyte growth factor; members of the bone morphogenetic proteins (BMP's) BMP-1, BMP-3, BMP-2; OP-1, BMP-2A, BMP-2B, or BMP-7; HBGF-1 or HBGF-2; growth differentiation factors (GDF's); members of the hedgehog family of proteins, including indian, sonic and desert hedgehog; ADMP-1; other members of the interleukin (IL) family; or members of the colony-stimulating factor (CSF) family, including CSF-1, G-CSF, and GM-CSF, or isoforms thereof; or VEGF, NELL-1 (neural epidermal growth factor-like 1), CD-RAP (cartilage-derived retinoic acid-sensitive protein) or combinations thereof.

In some embodiments, in addition to the chemical denervation agent, the device comprises a chemical denervation agent and growth factors (e.g., osteogenic protein). Exemplary osteogenic proteins include, but are not limited to, OP-1, OP-2, OP-3, BMP-2, BMP-3, BMP-3b, BMP-4, BMP-5, BMP-6, BMP-9, BMP-10, BMP-11, BMP-12, BMP-13, BMP-14, BMP-15, GDF-1, GDF-2, GDF-3, GDF-5, GDF-6, GDF-7, GDF-8, GDF-9, GDF-10, GDF-11, GDF-12, CDMP-1, CDMP-2, CDMP-3, DPP, Vg-1, Vgr-1, 60A protein, NODAL, UNIVIN, SCREW, ADMP, NEURAL, and TGF-beta. As used herein, the terms “morphogen,” “bone morphogen,” “BMP,” “osteogenic protein” and “osteogenic factor” embrace the class of proteins typified by human osteogenic protein 1 (hOP-1) and are described in U.S. Pat. No. 7,572,440. The entire disclosure is hereby incorporated by reference in the present disclosure.

Exemplary growth factors include, but are not limited to, members of the transforming growth factor beta family, including bone morphogenetic protein 2 (BMP-2); bone morphogenetic protein 4 (BMP-4); and transforming growth factors beta-1, beta-2, and beta-3 (potent keratinocyte growth factors). Other useful members of the transforming growth factor beta family include BMP-3, BMP-5, BMP-6, BMP-9, DPP, Vg1, Vgr, 60A protein, GDF-1, GDF-3, GDF-5, GDF-6, GDF-7, CDMP-1, CDMP-2, CDMP-3, BMP-10, BMP-11, BMP-13, BMP-15, Univin, Nodal, Screw, ADMP, Neural, and amino acid sequence variants thereof. Other growth factors include epidermal growth factor (EGF), which induces proliferation of both mesodermal and ectodermal cells, particularly keratinocytes and fibroblasts; platelet-derived growth factor (PDGF), which exerts proliferative effects on mesenchymal cells; fibroblast growth factor (FGF), both acidic and basic; and insulin-like growth factor 1 (IGF-1) or 2 (IGF-2), which mediate the response to growth hormone, particularly in bone growth. Further growth factors include osteogenic proteins. A particularly preferred osteogenic protein is OP-1, also known as bone morphogenetic protein 7 (BMP-7). OP-1 is a member of the transforming growth factor beta gene superfamily. It is a 139 amino acid residue long homodimer of MW 36,000. OP-1 induces new bone formation in vivo and promotes the repair of diaphyseal segmental bone defects and is described in U.S. Pat. No. 7,132,098. The entire disclosure is hereby incorporated by reference in the present disclosure.

In some embodiments, the therapeutic agent can comprise cells. Suitable cells include, without limitation, mesenchymal stem cells, periosteal cells, pluripotent stem cells, embryonic stem cells, osteoprogentior cells, osteoblasts, osteoclasts, bone marrow-derived cell lines, or any combination thereof. Other therapeutic agents include, for example, DNA, RNA, and their derivatives; vehicles for gene therapy, agents for inducing cell differentiation or de-differentiation or the like.

The therapeutic agent may also comprise nutrients such as chondroitin sulfate and/or glucosamine. The therapeutic agent can also include a lubricant including, but not limited to, lubricin, polyethylene glycol, or any combinations thereof.

In one embodiment, the therapeutic agent in the depot includes a chemical denervation agent, an anti-inflammatory, an anti-apoptotic, a proliferative agent, a fibrosis initiating agent, a differentiating agent, a gene therapy agent, a lubricating agent, a nutrient, a hygroscopic agent filler material, or a combination thereof.

A depot contains one or more therapeutic agent(s), as discussed above. A “depot” includes but is not limited to capsules, coatings, matrices, wafers, sheets, strips, ribbons, pills, pellets, microspheres, or other pharmaceutical delivery system or a combination thereof. Suitable materials for the depot are ideally pharmaceutically acceptable biodegradable and/or any bioabsorbable materials that are preferably FDA approved or GRAS materials. These materials can be polymeric or non-polymeric, as well as synthetic or naturally occurring, or a combination thereof. Typically, the depot will be a solid or semi-solid formulation comprising a biocompatible material that can be biodegradable. The term “solid” is intended to mean a rigid material, while “semi-solid” is intended to mean a material that has some degree of flexibility, thereby allowing the depot to bend and conform to the surrounding tissue requirements.

Suitable drug depots useful in the present application are described in U.S. Serial No. 12/105,474 filed Apr. 18, 2008 and published as U.S. Publication No. 20090263489, and U.S. Ser. No. 12/396,122, filed Mar. 2, 2009 and published as US20090263459. The entire disclosure of these applications is incorporated by reference herein in their entirety.

The drug depot may be microspheres or contain microspheres. Microspheres include generally spherical particles about 10 microns to about 2000 microns, or 10 microns to 1000 microns, or 50 microns to 250 microns and at least a population of microspheres in a diameter permitting parenteral administration. The process used to make the microspheres can be controlled to achieve a particular desired size range of microspheres. Other methods, such as sieving, can be used to more tightly control the size range of the microspheres.

In some embodiments, the drug depot comprises microspheres of a size range of from about 100 to 400 microns, which is well suited for delivery to the target tissue sites.

Microspheres comprise a hollow space encapsulated by lipids, polymers, or at least one surfactant, or any combination thereof, wherein the hollow space comprises a therapeutic agent (e.g., chemical denervation agent). In different embodiments, microspheres may include microbubbles or liposomes.

In some embodiments, the microspheres contain the therapeutic agent (e.g., chemical denervation agent) and can comprise a polymer, without limitation, poly(alpha-hydroxy acid), polyhydroxybutyric acid, polycaprolactone, poly(propylene fumarate), PEG, polyorthoester, polyanhydride, polyvinyl alcohol and ethylenevinyl acetate, or the like or combinations or copolymers thereof. In some embodiments, the microsphere can be derived from a poly(alpha-hydroxy acid), in particular, from a poly(lactide) (“PLA”) or a copolymer of D,L-lactide and glycolide or glycolic acid, such as a poly(D,L-lactide-co-glycolide) (“PLG” or “PLGA”), or a copolymer of D,L-lactide and caprolactone. The microspheres may be derived from any of various polymeric starting materials which have a variety of molecular weights and, in the case of the copolymers such as PLG, a variety of lactide: glycolide ratios, the selection of which will be largely a matter of choice, depending in part on the desired dose of the active ingredient(s).

In some embodiments, the microspheres containing the chemical denervation agent, as well as other therapeutic agents, are loaded into the formulation and are disposed uniformly throughout it or in a particular region (e.g., center or borders) and delivered in, at, or near the basivertebral nerve. The microspheres will degrade and release the therapeutic agent at, near or in the basivertebral nerve and the microspheres will begin releasing the therapeutic agent immediately and/or in a sustained release fashion to the desired tissue location.

The drug depot comprises a therapeutically effective amount of the denervation agent, as well as the therapeutic agent. A “therapeutically effective amount” or “effective amount” is such that when administered, the drug results in alteration of the biological activity, such as, for example, the ablation of the nerve or the inhibition of inflammation, reduction or alleviation of pain, improvement in the condition through muscle relaxation, degradation of a portion of the vertebral nerve, etc.

In some embodiments the formulation of the drug depot is designed for immediate release. In other embodiments the formulation is designed for sustained release. In other embodiments, the formulation comprises one or more immediate release surfaces or layers and one or more sustain release surfaces or layers in one depot.

The phrases “sustained release” or “sustain release” (also referred to as extended release or controlled release) are used herein to refer to one or more therapeutic agent(s) that is introduced into the body of a human or other mammal and continuously or continually releases a stream of one or more therapeutic agents (e.g., chemical denervation agents) over a predetermined time period and at a therapeutic level sufficient to achieve a desired therapeutic effect throughout the predetermined time period. Reference to a continuous or continual release stream is intended to encompass release that occurs as the result of biodegradation in vivo of the drug depot, or a matrix or component thereof, or as the result of metabolic transformation or dissolution of the therapeutic agent(s) or conjugates of therapeutic agent(s). As persons of ordinary skill are aware, sustained release formulations may, by way of example, be created as films, slabs, pellets, microparticles, microspheres, microcapsules, spheroids, shaped derivatives or pastes. Further, the formulations may be used in conjunction with any implantable or insertable system that a person of ordinary skill would appreciate as useful in connection with embodiments herein including but not limited to parenteral formulations, microspheres, microcapsules, pastes, implantable rods, pellets, plates or fibers, etc. The chemical denervation agent can be in the device as a sustained release formulation, where one or more regions or layers of the device release the chemical denervation agent into the vertebrae (or other places where denervation of the spine is required to treat chronic back pain so as to degrade the vertebral nerve over an extended period of time (e.g., 3 months to 1 year).

The immediate release therapeutic agent such as the chemical denervation agent can be released first then either followed by a second therapeutic agent or nothing at all. The phrase “immediate release” is used herein to refer to one or more therapeutic agent(s) that is introduced into the body and that is allowed to dissolve in or become absorbed at the location to which it is administered, with no intention of delaying or prolonging the dissolution or absorption of the drug. Immediate release refers to the release of drug within a short time period following administration, e.g., generally within a few seconds or minutes to about 1, 2, 3, 4, 5, 6, 7, 8, 9, 10, 11, 12 hours or within 24 hours after implantation. The chemical denervation agent can be in the device as an immediate release formulation, where one or more regions or layers of the device release the chemical denervation agent into the vertebrae to degrade the basivertebral nerve. The immediate release region or layer of the device can be in liquid solutions, suspensions, or emulsions forms or semi-solid or solid forms having a suitable excipient for immediate release. Suitable excipients are, for example, water, saline, dextrose, glycerol, ethanol, pH buffering agents, metal ion salts, or other such buffers. The formulation also may contain other minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate, cyclodextrins or a combination thereof.

For example, an immediate release formulation of a chemical denervation agent that can be incorporated into a drug depot can be hyaluronidase formulated with one or more of EDTA, NaCl, CaCl₂, histidine, lactose, albumin, Pluronic® F68, TWEEN® and/or other detergent or other similar agents. For example, compositions provided herein can contain one or more pH buffers (such as, for example, histidine, phosphate, or other buffers), or acidic buffer (such as acetate, citrate, pyruvate, Gly-HCl, succinate, lactate, maleate or other buffers), tonicity modifier (such as, for example, an amino acid, polyalcohol, NaCl, trehalose, other salts and/or sugars), stabilizer, chelating agent, such as ethylenediaminetetraacetic acid, ethylenediaminetetraacetate or calcium EDTA, oxygen scavenger, such as methionine, ascorbic acid/ascorbate, citric acid/citrate, or albumin, and/or a preservative, such as preservative containing an aromatic ring (e.g. phenol or cresol). In some embodiments, the depot does not contain any preservatives and, therefore, is preservative free.

Exemplary stabilizers that are useful for the depot containing the chemical denervation agent include, for example, polysorbates or proteins such as human serum albumin.

The depot can be designed to provide the desired release rate profile for immediate release and/or sustained release of the chemical denervation agent and then a therapeutic agent, analgesic, anti-inflammatory agent, growth factor, etc.). The phrase “release rate profile” refers to the percentage of active ingredient that is released over fixed units of time, e.g., mcg/hr, mcg/day, mg/hr, mg/day, 10% per day for ten days, and the like. As persons of ordinary skill know, a release rate profile may be but need not be linear. By way of a non-limiting example, the drug depot may be a pellet that releases at least one chemical denervation agent, analgesic, anti-inflammatory agent, growth factor, and/or analgesic agent in a bolus dose and at least one chemical denervation agent, analgesic, anti-inflammatory agent, and/or growth factor over an extended period of time (e.g., 3 days to 3 months).

The depot can be biodegradable. The term “biodegradable” includes that all or parts of the drug depot will degrade over time by the action of enzymes, by hydrolytic action and/or by other similar mechanisms in the human body. In various embodiments, “biodegradable” includes that the depot can break down or degrade within the body to non-toxic components after or while a therapeutic agent has been or is being released. By “bioerodible” it is meant that the depot will erode or degrade over time due, at least in part, to contact with substances found in the surrounding tissue, fluids or by cellular action. By “bioabsorbable” it is meant that the depot will be broken down and absorbed within the human body, for example, by a cell or tissue. “Biocompatible” means that the depot will not cause substantial tissue irritation or necrosis at the target tissue site.

The depot may comprise non-biodegradable material. Examples of non-biodegradable polymers include, but are not limited to, various cellulose derivatives (carboxymethyl cellulose, cellulose acetate, cellulose acetate propionate, ethyl cellulose, hydroxypropyl methyl cellulose, hydroxyalkyl methyl celluloses, and alkyl celluloses), silicon and silicon-based polymers (such as polydimethylsiloxane), polyethylene-co-(vinyl acetate), poloxamer, polyvinylpyrrolidone, poloxamine, polypropylene, polyamide, polyacetal, polyester, poly ethylene-chlorotrifluoroethylene, polytetrafluoroethylene (PTFE or “Teflon™”), styrene butadiene rubber, polyethylene, polypropylene, polyphenylene oxide-polystyrene, poly-α-chloro-p-xylene, polymethylpentene, polysulfone, non-degradable ethylene-vinyl acetate (e.g., ethylene vinyl acetate disks and poly(ethylene-co-vinyl acetate)), and other related biostable polymers.

Non-resorbable polymers can also include, but are not limited to, delrin, polyurethane, copolymers of silicone and polyurethane, polyolefins (such as polyisobutylene and polyisoprene), acrylamides (such as polyacrylic acid and poly(acrylonitrile-acrylic acid)), neoprene, nitrile, acrylates (such as polyacrylates, poly(2-hydroxy ethyl methacrylate), methyl methacrylate, 2-hydroxyethyl methacrylate, and copolymers of acrylates with N-vinyl pyrrolidone), N-vinyl lactams, polyacrylonitrile, glucomannan gel, vulcanized rubber and combinations thereof. Examples of polyurethanes include thermoplastic polyurethanes, aliphatic polyurethanes, segmented polyurethanes, hydrophilic polyurethanes, polyether-urethane, polycarbonate-urethane and silicone polyether-urethane. Other suitable non-resorbable material include, but are not limited to, lightly or highly cross-linked biocompatible homopolymers and copolymers of hydrophilic monomers such as 2-hydroxyalkyl acrylates and methacrylates, N-vinyl monomers, and ethylenically unsaturated acids and bases; polycyanoacrylate, polyethylene oxide-polypropylene glycol block copolymers, polygalacturonic acid, polyvinyl pyrrolidone, polyvinyl acetate, polyalkylene glycols, polyethylene oxide, collagen, sulfonated polymers, vinyl ether monomers or polymers, alginate, polyvinyl amines, polyvinyl pyridine, and polyvinyl imidazole. Depending on the amount of crosslinking within the bioresorbable polymers, the degradation time of the polymer can be reduced, thus making the polymer, for the purpose of this application, appear to be non-resorbable over the time frame of the use of the material for this invention.

The drug depot can provide the appropriate pain management medication. The phrase “pain management medication” includes one or more therapeutic agents that are administered to prevent, alleviate or remove pain entirely. These include anti-inflammatory agents, analgesics, anesthetics, narcotics, and so forth, or combinations thereof.

In various embodiments, the depot can be designed to cause an initial burst dose of one or more therapeutic agents within the first 24 hours after implantation. “Initial burst” or “burst effect” or “bolus dose” or “pulse dose” refer to the release of therapeutic agent from the depot during the first 24 hours after the depot comes in contact with an aqueous fluid (e.g., fluid in the vertebrae). The burst effect may be an immediate release of the chemical denervation agent. The “burst effect” is believed to be due to the increased release of therapeutic agent in particular the chemical denervation agent from the depot. The initial burst effect or bolus dose may be determined beforehand by formulating the depot by calculating the quotient obtained by dividing (i) the effective amount by weight of therapeutic agent to be released from the depot or region in a predetermined initial period of time after implantation of the depot, by (ii) the total amount of therapeutic agent that is to be delivered from an implanted composition. It is understood that the initial burst may vary depending on the shape and surface area of the implant.

The burst effect with respect to the region of the depot or individual depot, in various embodiments, can be designed so that a larger initial dose may be released over a short period of time to achieve the desired effect. For example, if a drug depot is designed to release an immediate amount and then a sustained amount of chemical denervation, then the initial burst dose or bolus dose region or depot will be designed to release a percentage of the dose within the first 24 hours (e.g., 10 mg of chemical denervation agent or 66% of the 48 hour dose within 24 hours). Thus, the burst effect of the drug depot or region of the drug depot releases more therapeutic agent than the sustained release region or depot.

A region or depot that utilizes a burst effect or bolus dose will release more therapeutic agent (e.g., chemical denervation agent, analgesic, anti-inflammatory, and/or growth factor) than the sustained release region or depot. For example, particularly with painful conditions such as discogenic back pain, or the like, the initial burst effect of the drug depot or region of the drug depot will be advantageous as it will provide more immediate pain and/or inflammation relief as a bolus dose of drug will be released at or near the target tissue site and provide the desired reducing, or alleviation of signs or symptoms of pain and/or inflammation. For example, the drug depot or region of the drug depot may release 51%, 52%, 53%, 54%, 55%, % 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% of the desired dose within the first one to twelve hours to ablate the basivertebral nerve, reduce, prevent or treat pain and/or inflammation.

In some embodiments, the drug depot may have an initial burst effect to release the drug shortly after it is implanted. Various factors can be adjusted to achieve the initial burst of therapeutic agent release. First, the initial burst can be controlled by factors related to the property of the depot, such as the water immiscibility of the solvent, polymer/solvent ratio, and the property of the polymer. The extent of water immiscibility of the solvent used in the depot affects that rate aqueous body fluid can penetrate the depot to release the therapeutic agent. Generally, higher water solubility leads to a higher initial burst while water immiscibility leads to a lower initial burst or slower release (sustained release) of the therapeutic agent.

Suitable solvents that can be used to control initial burst release or sustained release include, but are not limited to, methyl benzoate, ethyl benzoate, n-propyl benzoate, isopropyl benzoate, butyl benzoate, isobutyl benzoate, sec-butyl benzoate, tert-butyl benzoate, isoamyl benzoate, benzyl benzoate, water, alcohol, low molecular weight PEG (less than 1,000 MW), triacetin, diacetin, tributyrin, triethyl citrate, tributyl citrate, acetyl triethyl citrate, acetyl tributyl citrate, triethylglycerides, triethyl phosphate, diethyl phthalate, diethyl tartrate, mineral oil, polybutene, silicone fluid, glycerin, ethylene glycol, octanol, ethyl lactate, propylene glycol, propylene carbonate, ethylene carbonate, butyrolactone, ethylene oxide, propylene oxide, N-methyl-2-pyrrolidone, 2-pyrrolidone, glycerol formal, methyl acetate, ethyl acetate, methyl ethyl ketone, dimethylformamide, glycofurol, dimethyl sulfoxide, tetrahydrofuran, caprolactam, decylmethylsulfoxide, oleic acid, 1-dodecylazacyclo-heptan-2-one, or mixtures thereof. The solvent can be mixed, in various embodiments, with the therapeutic agent and/or polymers to obtain the desired release profile.

Further, varying the molecular weight of the polymer in the depot, or adjusting the molecular weight distribution of the polymer material in the depot vehicle can affect the initial burst and the release rate of therapeutic agent from the depot. Generally, a higher molecular weight polymer renders a lower initial burst and slower release rate of the therapeutic agent. The polymers may have different end groups such as acid and ester end groups. As persons of ordinary skill in the art are aware, when implantable elastomeric depot compositions having a blend of polymers with different end groups are used, the resulting formulation will have a lower burst index and a regulated duration of delivery. For example, one may use polymers with acid (e.g., carboxylic acid) and ester end groups (e.g., methyl of ethyl ester end groups).

Additionally, by varying the comonomer ratio of the various monomers that form a polymer (e.g., the L/G (lactic acid/glycolic acid) or G/CL (glycolic acid/polycaprolactone) ratio for a given polymer) there will be a resulting depot composition having a regulated burst index and duration of delivery. For example, a depot composition having a polymer with a L/G ratio of 50:50 may have a short duration of delivery ranging from about two days to about one month; a depot composition having a polymer with a L/G ratio of 65:35 may have a duration of delivery of about two months; a depot composition having a polymer with a L/G ratio of 75:25 or L/CL ratio of 75:25 may have a duration of delivery of about three months to about four months; a depot composition having a polymer ratio with a L/G ratio of 85:15 may have a duration of delivery of about five months; a depot composition having a polymer with a L/CL ratio of 25:75 or PLA may have a duration of delivery greater than or equal to six months; a depot composition having a terpolymer of CL/G/L with G greater than 50% and L greater than 10% may have a duration of delivery of about one month and a depot composition having a terpolymer of CL/G/L with G less than 50% and L less than 10% may have a duration months up to six months. In general, increasing the G content relative to the CL content shortens the duration of delivery whereas increasing the CL content relative to the G content lengthens the duration of delivery. Thus, among other things, depot compositions having a blend of polymers having different molecular weights, end groups and comonomer ratios can be used to create a depot formulation having a lower burst index and a regulated duration of delivery.

Factors such as the particle size, the disintegration of the particulates, the morphology of the particulates (e.g., whether pores are present in the particulates before implanting or can be formed easily by body fluid attack), coatings, complex formation by the therapeutic agent and the strength of complex bond, can be manipulated to achieve the desired low initial burst and release rate.

The drug depot may comprise at least one analgesic agent or its pharmaceutically acceptable salt. Examples of analgesic agents include but are not limited to acetaminophen, a local anesthetic, such as for example, lidocaine, bupivacaine, ropivacaine, opioid analgesics such as amitriptyline, carbamazepine, gabapentin, pregabalin, clonidine, opioid analgesics or a combination thereof. Opioid analgesics include, alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphene, normorphine, norpipanone, opium, oxycodone, oxymorphone, papavereturn, pentazocine, phenadoxone, phenomorphan, phenazocine, phenoperidine, piminodine, piritramide, propheptazine, promedol, properidine, propoxyphene, sufentanil, tilidine, tramadol or a combination thereof. Analgesic agents also include those with analgesic and anti-inflammatory properties, such as, for example, amitriptyline, carbamazepine, gabapentin, pregabalin, clonidine, or a combination thereof.

In some embodiments, the drug depot contains anti-inflammatory agents and/or analgesic comprising flurbiprofen, indoprofen, naproxol, pentazocine, proxazole, tramadol, verilopam, volazocine, xylazine, zucapsaicin, phenyhydantoin, phenobarbital, primidone, ethosuximide, methsuximide, phensuximide, trimethadione, diazepam, benzodiazepines, phenacemide, pheneturide, acetazolamide, sulthiame, bromide, nalorphine, naloxone, naltrexone, salycilates, phenylbutazone, indomethacin, phenacetin, dextropropoxyphene, levomethadyl, pethidine, remifentanil, flupirtine or a combination thereof.

In some embodiments, the anti-inflammatory and/or analgesic agents include, but are not limited to, salicylates, diflunisal, indomethacin, ibuprofen, naproxen, tolmetin, ketorolac, diclofenac, ketoprofen, fenamates (mefenamic acid, meclofenamic acid), enolic acids (piroxicam, meloxicam), nabumetone, celecoxib, etodolac, nimesulide, apazone, gold, sulindac or tepoxalin; antioxidants, such as dithiocarbamate, and other compounds such as sulfasalazine [2-hydroxy-5-[-4-[C2-pyridinylamino)sulfonyl]azo]benzoic acid], steroids, such as fluocinolone, cortisol, cortisone, hydrocortisone, fludrocortisone, prednisone, prednisolone, methylprednisolone, triamcinolone, betamethasone, dexamethasone, beclomethasone, fluticasone, protein inhibitors of TNF, such as etanercept, Remicade, IL-1, such as Kineret®, p38, RANK, RANKL or a combination thereof.

The drug depot can comprise at least one analgesic agent or its pharmaceutically acceptable salt and/or at least one anti-inflammatory agent or its pharmaceutically acceptable salt and may be co-administered with a muscle relaxant. Co-administration may involve administering at the same time in separate drug depots or formulating together in the same drug depot.

Exemplary muscle relaxants include by way of example and not limitation, alcuronium chloride, atracurium bescylate, baclofen, carbolonium, carisoprodol, chlorphenesin carbamate, chlorzoxazone, cyclobenzaprine, dantrolene, decamethonium bromide, fazadinium, gallamine triethiodide, hexafluorenium, meladrazine, mephensin, metaxalone, methocarbamol, metocurine iodide, pancuronium, pridinol mesylate, styramate, suxamethonium, suxethonium, thiocolchicoside, tizanidine, tolperisone, tubocuarine, vecuronium, or combinations thereof.

The drug depot may also comprise other therapeutic agents or active ingredients in addition to the at least one chemical denervation agent, the at least one analgesic agent or its pharmaceutically acceptable salt or the at least one anti-inflammatory agent or its pharmaceutically acceptable salt. Suitable additional therapeutic agents include, but are not limited to, integrin antagonists, alpha-4 beta-7 integrin antagonists, cell adhesion inhibitors, interferon gamma antagonists, CTLA4-Ig agonists/antagonists (BMS-188667), CD40 ligand antagonists, Humanized anti-IL-6 mAb (MRA, Tocilizumab, Chugai), HMGB-1 mAb (Critical Therapeutics Inc.), anti-IL2R antibodies (daclizumab, basilicimab), ABX (anti IL-8 antibodies), recombinant human IL-10, or HuMax IL-15 (anti-IL 15 antibodies).

Other suitable therapeutic agents that may be co-administered or in the depot with the chemical denervation agent, anti-inflammatory agent or analgesic agent include IL-1 inhibitors, such Kineret® (anakinra) which is a recombinant, non-glycosylated form of the human interleukin-1 receptor antagonist (IL-1Ra), or AMG 108, which is a monoclonal antibody that blocks the action of IL-1. Therapeutic agents also include excitatory amino acids such as glutamate and aspartate, antagonists or inhibitors of glutamate binding to NMDA receptors, AMPA receptors, and/or kainate receptors. It is contemplated that where desirable a pegylated form of the above may be used. Examples of other therapeutic agents include NF kappa B inhibitors such as glucocorticoids, antioxidants, such as dithiocarbamate.

In some embodiments, the at least one biodegradable polymer comprises poly(lactic-co-glycolic acid) (PLA) or poly(orthoester) (POE) or a combination thereof. The poly(lactic-co-glycolic acid) may comprise a mixture of polyglycolide (PGA) and polylactide and in some embodiments, in the mixture, there is more polylactide than polyglycolide. In various other embodiments there is 100% polylactide and 0% polyglycolide; 95% polylactide and 5% polyglycolide; 90% polylactide and 10% polyglycolide; 85% polylactide and 15% polyglycolide; 80% polylactide and 20% polyglycolide; 75% polylactide and 25% polyglycolide; 70% polylactide and 30% polyglycolide; 65% polylactide and 35% polyglycolide; 60% polylactide and 40% polyglycolide; 55% polylactide and 45% polyglycolide; 50% polylactide and 50% polyglycolide; 45% polylactide and 55% polyglycolide; 40% polylactide and 60% polyglycolide; 35% polylactide and 65% polyglycolide; 30% polylactide and 70% polyglycolide; 25% polylactide and 75% polyglycolide; 20% polylactide and 80% polyglycolide; 15% polylactide and 85% polyglycolide; 10% polylactide and 90% polyglycolide; 5% polylactide and 95% polyglycolide; and 0% polylactide and 100% polyglycolide.

In various embodiments that comprise both polylactide and polyglycolide; there is at least 95% polylactide; at least 90% polylactide; at least 85% polylactide; at least 80% polylactide; at least 75% polylactide; at least 70% polylactide; at least 65% polylactide; at least 60% polylactide; at least 55%; at least 50% polylactide; at least 45% polylactide; at least 40% polylactide; at least 35% polylactide; at least 30% polylactide; at least 25% polylactide; at least 20% polylactide; at least 15% polylactide; at least 10% polylactide; or at least 5% polylactide; and the remainder of the biopolymer being polyglycolide.

In some embodiments, the biodegradable polymer comprises at least 10 wt. %, at least 50 wt. %, at least 60 wt. %, at least 70 wt. %, at least 80 wt. %, at least 85 wt. %, at least 90 wt. %, at least 95 wt. %, or at least 99 wt. % of the formulation. In some embodiments, the at least one biodegradable polymer and the denervation agent are the only components of the pharmaceutical formulation that is used to make the depot.

In some embodiments, the methods provided can be used to treat patients with mild to moderate degeneration of the vertebrae so that two end plates of the vertebrae collapse towards each other putting pressure on the basivertebral nerve. As explained herein, delivery of the chemical denervation agent may be accomplished with little or no additional injury to the patient. In some embodiments, the methods provided herein may be especially useful for patients that are not good candidates for other treatments, such as, surgery, spinal fixation, vertebrae replacement, spinal fusion, and other surgical regimens for the treatment of degenerated vertebral disease.

Accordingly, in some embodiments, there is a method for treating an chronic back pain by chemical denervation of the basiverebral nerve, the method comprising administering an implantable and biodegradable device to the effective area comprising the basivertebral nerve, the device comprising a chemical denervation agent to proteolytically degrade at least a portion of the basivertebral nerve and being configured to immediately release an effective amount of the chemical denervation agent within 24 hours and provide sustained release of the chemical denervation agent or/and a therapeutic device over a period of up to one year to treat the degenerative vertebral disease.

In some embodiments, the method utilizes an implantable and biodegradable device that is administered to the effective area containing the vertebral nerve by inserting a needle or cannula into the vertebrae such that the inserted end of the needle or cannula is inside the vertebrae; and the device is pushed into the body if the vertebrae and then the needle or cannula is removed.

In some embodiments, the method utilizes an implantable and biodegradable device that is a drug depot comprising a polymer and chemical denervation agent such as hyaluronidase to proteolytically degrade at least a portion of a nerve of the vertebrae, the drug depot having an immediate release layer is configured to release an effective amount of the hyaluronidase within 24 hours and a sustained release layer contacting the immediate release layer to provide sustained release of the hyaluronidase over a period of up to 3 months to treat the chronic back pain.

A skilled artisan will be capable of determining the desired amount of chemical denervation agent based on a number of factors, including, for example, the degree of vertebrae/disc degeneration, the age, weight, and health of the patient, and the degree of restoration required. Additionally, the methods provided herein may be used to slow the rate of progressive collapse of vertebrae and/or maintain the height of a vertebrae experiencing progressive collapse.

### Cannula or Needle

The chemical denervation agent can be loaded in a cannula or needle that is designed to cause minimal physical and psychological trauma to the patient. Cannulas or needles include tubes that may be made from materials, such as for example, polyurethane, polyurea, polyether(amide), PEBA, thermoplastic elastomeric olefin, copolyester, and styrenic thermoplastic elastomer, steel, aluminum, stainless steel, titanium, metal alloys with high non-ferrous metal content and a low relative proportion of iron, carbon fiber, glass fiber, plastics, ceramics or combinations thereof. The cannula or needle may optionally include one or more tapered regions. In various embodiments, the cannula or needle may be beveled. The cannula or needle may also have a tip style vital for accurate treatment of the patient depending on the site for implantation. Examples of tip styles include, for example, Trephine, Cournand, Veress, Huber, Seldinger, Chiba, Francine, Bias, Crawford, deflected tips, Hustead, Lancet, or Tuohey. In various embodiments, the cannula or needle may also be non-coring and have a sheath covering it to avoid unwanted needle sticks.

The dimensions of the hollow cannula or needle, among other things, will depend on the site for implantation. For example, the width of the epidural space is only about 3-5 mm for the thoracic region and about 5-7 mm for the lumbar region. Thus, the needle or cannula, in various embodiments, can be designed for these specific areas. Some examples of lengths of the cannula or needle may include, but are not limited to, from about 50 to 150 mm in length, for example, about 65 mm for epidural pediatric use, about 85 mm for a standard adult and about 150 mm for an obese adult patient. The thickness of the cannula or needle will also depend on the site of implantation. In various embodiments, the thickness includes, but is not limited to, from about 0.05 to about 1.655. The gauge of the cannula or needle may be the widest or smallest diameter or a diameter in between for insertion into a human or animal body. The widest diameter is typically about 14 gauge, while the smallest diameter is about 25 gauge. In various embodiments the gauge of the needle or cannula is about 17 to about 25 gauge.

In various embodiments, the plunger, cannula, and/or drug depot can include markings that indicate location at or near the site beneath the skin. Radiographic markers can be included to permit the user to accurately position the needle or cannula, or drug depot into the site of the patient. These radiographic markers will also permit the user to track movement and degradation of the drug depot at the site over time. In this embodiment, the user may accurately position the drug depot in the site using any of the numerous diagnostic-imaging procedures. Such diagnostic imaging procedures include, for example, X-ray imaging or fluoroscopy. Examples of such radiographic markers include, but are not limited to, barium, calcium phosphate, and/or metal beads.

In various embodiments, the needle or cannula may include a transparent or translucent portion that can be visualizable by ultrasound, fluoroscopy, x-ray, or other imaging techniques. In such embodiments, the transparent or translucent portion may include a radiopaque material or ultrasound responsive topography that increases the contrast of the needle or cannula relative to the absence of the material or topography. The drug depot may be administered in conjunction with a standard discogram.

In one embodiment, the delivery system for the drug depot can include any syringe based system that would be used to administer the denervation agent to the effective treatment zone containing the vertebral nerve. These syringe based systems include inflation syringes with a fine and coarse drive, in conjunction with a pressure gage.

In one embodiment, the drug depot or chemical denervation agent is administered to the treatment zone including the nerve in vertebrae using a Kyphon Discyphor catheter system available from Medtronic Spine LLC in Sunnyvale, Calif., USA, where the damaged vertebrae can be diagnosed and treated using the same catheter. Thus, the drug depot can be delivered to the vertebrae in one procedure using the same catheter system.

### Administration

Referring to FIG. 1, the reference numeral 12 refers to a vertebral body. Vertebral body 12 includes a bottom endplate 16 and a top endplate 18. Basivertebral nerve 10 is embedded in vertebral body 12 and includes nerve branches 14. Nerve branches 14 branch up to the surface 8 of the top and bottom endplates, exposing it to the stresses placed on the vertebral body.

Referring now to FIG. 2, an embodiment of treating degenerative vertebrae or trauma stricken vertebrae by delivering chemical denervation agents directly to the area containing the nerve. In the embodiments of the present application, a cannula 30 and needle 32 are inserted into the vertebral body 12 through opening 33 to deliver a chemical denervation (not shown) agent to an effective treatment zone 22 at, near, or in the damaged area of the vertebra. The direct delivery of the chemical denervation to the treatment zone will ablate the basivertebral nerve 10.

Referring now to FIG. 3, an embodiment of treating degenerative vertebrae or trauma stricken vertebrae by delivering chemical denervation agents directly to the area containing the nerve. In this embodiment of the present disclosure, syringe 46 is attached to needle 38 to administer a chemical denervation agent 44 into the effective treatment zone 22 of the vertebrae to ablate basivertebral nerve 10.

In some embodiments, the vertebral body is accessed using a posterior unilateral, bilateral or multi-lateral approach. In alternative embodiments, the vertebral body may be accessed with a lateral approach, an anterior approach, a trans-pedicular/vertebral endplate approach, an axial approach, or any other suitable vertebral body accessing approach.

Referring now to FIG. 4, another embodiment of chronic back pain treatment. In the embodiments of the present application, a chemical denervation agent in a device (e.g. drug depot 48) is to be delivered through the vertebral body by inserting a needle 38 into the cannula 32 through the opening 33 delivering the drug depot containing the chemical denervation agent to the treatment zone 22. The chemical denervation agent can be delivered by coupling a syringe containing this agent to needle 38. The chemical denervation agent will be released from the drug depot, which will degrade or cause the dissolution of the basivertebral nerve, thereby reducing the chronic back pain of the patient. The drug depot is configured to immediately release the chemical denervation agent within seconds or minutes to within 24 hours so that the chemical denervation agent begins to exert its effect. Drug depot 48 is also configured to provide sustained release of the chemical denervation agent as the drug depot degrades, where it will release the chemical denervation agent into the treatment zone for extended periods of time. In this way, the chemical denervation agent will stay locally at the target tissue site and will provide treatment of the vertebrae for an extended period of time. Thus, one catheter can be used to deliver an “all-in-one composition”.

Referring now to FIG. 5, in this embodiment of the present disclosure, an enlarged view of a solid or semi-solid drug depot 50 containing an immediate release layer 52 that immediately releases a chemical denervation agent as soon as it is implanted within 24 hours and, as the layer degrades, a sustained release layer 54 releases the chemical denervation agent over a prolonged period of time (e.g., about 3 days to about 3 months or longer).

Alternatively, the drug depots can be designed with regions that provide immediate release of the same or different therapeutic agent and regions that provide sustain release of the same or different therapeutic agent.

In some embodiments, the drug depot may have one or more additional therapeutic agents disposed in one or more regions that can provide immediate or sustain release of the therapeutic agent. For example, a growth factor, an analgesic, an anti-inflammatory agent or a combination thereof can be disposed in layer 51 and can be released either in an immediate release or a sustained release fashion after the immediate release layer comprising the chemical denervation agent is released.

In the embodiment shown, the additional therapeutic layer 51 is separate from the immediate release layer 52 and can comprise an anti-inflammatory agent or analgesic that can be in an immediate release formulation to provide immediate relief of pain and/or inflammation locally at the site of implantation. After the additional therapeutic agent is released, a sustained release layer 54 containing the chemical denervation agent can release the chemical denervation agent over an extended period of time. After this layer degrades, an additional therapeutic agent shown as layer 53, such as for example, a growth factor is kept separate from the sustained release layer 54 containing the chemical denervation agent. This is because, in some embodiments, the chemical denervation agent, which is often an enzyme, can degrade the growth factor. By keeping the growth factor and the chemical denervation agent in a separate layer, premature degradation of the growth factor and/or chemical denervation agent and loss in potency is reduced. After the growth factor 53 is released from the depot and as the layer degrades, the sustained release layer can degrade and continue to release the chemical denervation agent over a prolonged period of time.

Although the drug depot 50 is shown as a five layered depot, it will be understood by one of ordinary skill in the art that the depot can have the chemical denervation agent and additional therapeutic agent disposed in the same or different layers in immediate release or sustained release formulations. In some embodiments, the drug depot can have an immediate release portion and a sustained release portion disposed uniformly distributed through one or more layers of the depot containing the chemical denervation agent alone or in combination with the additional therapeutic agent. In some embodiments, the drug depot can have one, two, three, four, five, six, seven, eight, nine, ten or more layers, where each layer can contain one or more therapeutic agents that can be in an immediate release formulation, sustained release formulation or a combination thereof. A multi-layered or multi-region depot is shown in FIG. 4.

FIG. 5 illustrates an enlarged view of a drug depot 56 containing an immediate release region 58 which is in liquid, semi-solid or solid form that immediately releases a chemical denervation agent as soon as it is implanted within 24 hours and, as the region degrades, a sustained release microspheres (one shown as 60) releases the chemical denervation agent over a prolonged period of time (e.g., about 3 days to about 3 months or longer).

In some embodiments, the drug depot may have one or more additional therapeutic agents disposed in one or more regions that can provide immediate or sustained release of the therapeutic agent. For example, a growth factor, an analgesic, an anti-inflammatory agent or a combination thereof can be disposed in region 58 or microsphere 60 and be released either in an immediate release or a sustained release fashion as the drug depot 56 degrades.

Although the drug depot 56 is shown as a square shape, it will be understood by one of ordinary skill in the art that the depot can be any shape or it can be amorphous and cure or harden as a depot or it can be a plurality of depots containing the chemical denervation agent in a sustained release formulation. In some embodiments, the drug depots can be uniformly disposed throughout the formulation or it can be concentrated in one area of the formulation.

The techniques and devices described herein provide a safe and effective means for various types of vertebrae/disc treatment including, but not limited to, chemical denervation, pain-management, repair, and regeneration.

In some embodiments, the therapeutically effective dosage amount (e.g., chemical denervation agent, analgesic, anti-inflammatory agent, and/or growth factor, etc.) and the release rate profile of the therapeutic agent is sufficient to reduce inflammation and/or pain for a period of at least one day, for example, 1-90 days, 1-10 days, 1-3 days, 3-7 days, 3-12 days; 3-14 days, 3-25 days, 3-45 days, 7-10 days, 7-14 days, 7-21 days, 7-30 days, 7-50 days, 7-90 days, 7-140 days, or 14-140 days, 3 days-3 months, 7 days to 6 months, 10 days to 1 year.

In some embodiments, the therapeutic agent is released from the depot as a bolus dose at the target tissue to provide an immediate release of the therapeutic agent.

In some embodiments, there is a composition useful for the treatment of pain and/or inflammation associated degenerative vertebrae comprising an effective amount of at least one chemical denervation agent alone or in combination with at least one analgesic agent, at least one anti-inflammatory agent, and/or at least one growth factor that is capable of being administered to a target tissue site e.g., a pain or inflammatory site. By way of example, they may be administered locally to one or more vertebrae.

In some embodiments, a plurality of depots containing the chemical denervation agent, analgesic, anti-inflammatory agent, and/or growth factor can be placed in and around the nerve to provide a strategy to triangulate around the pain generator. A strategy of triangulation may be effective when administering multiple depot pharmaceutical formulations. Thus, a plurality (at least two, at least three, at least four, at least five, at least six, at least seven, etc.) drug depots may be placed around the target tissue site (also known as the pain generator or pain generation site) such that the target tissue site falls within a region that is either between the formulations when there are two, or within an area whose perimeter is defined by a set of plurality of formulations. Alternatively repeat administration to lengthen the delivery timeframe may be required.

In some embodiments, a desired release rate profile is maintained for at least three days, at least ten days, at least twenty days, at least thirty days, at least forty days, at least fifty days, at least ninety days, at least one hundred days, at least one-hundred and thirty-five days, at least one-hundred and fifty days, or at least one hundred and eighty days, or at least 1 year.

In some embodiments, the drug depot may release 5%, 10%, 15%, 20%, 25%, 30%, 40%, 50%, 60%, 70%, 80%, 90%, 95%, or 99% of the therapeutic agent (e.g., chemical denervation agent) or pharmaceutically acceptable salt thereof relative to a total amount of the therapeutic agent loaded in the drug depot over a period of at least three days, at least seven days, at least ten days, at least twenty days, at least thirty days, at least forty days, at least fifty days, at least ninety days, at least one hundred days, at least one-hundred and thirty-five days, at least one-hundred and fifty days, or at least one hundred and eighty days, or at least 1 year.

In various embodiments, the chemical denervation agent, analgesic, anti-inflammatory agent, and/or growth factor will be released in an initial burst dose, then one or more of these therapeutic agents will be released daily for 3 days and then stop (e.g., this will be suitable to reduce, prevent or treat, acute pain), while the chemical denervation agent, analgesic, anti-inflammatory agent, and/or growth factor will be released daily without a burst dose for 3 to 12 days, 5 to 10 days or 7 to 10 days or longer after the drug depot is administered to the target tissue site.

In various embodiments, a kit is provided comprising one or more drug depots containing one or more chemical denervation agents, analgesics, anti-inflammatory agents, and/or growth factors. The kit may also include additional parts along with the drug depots combined together to be used to administer it. The kit may include the drug depot and delivery device in a first compartment. The second compartment may include a canister holding the drug depots and any other instruments needed for the localized drug delivery. A third compartment may include gloves, drapes, needles, wound dressings and other procedural supplies for maintaining sterility of the implanting process, as well as an instruction booklet. A fourth compartment may include additional needles and/or sutures. Each tool may be separately packaged in a plastic pouch that is radiation sterilized. A fifth compartment may include an agent for radiographic imaging. A cover of the kit may include illustrations of the implanting procedure and a clear plastic cover may be placed over the compartments to maintain sterility.

The devices and methods of the present application can be used to treat chronic back pain caused by degenerative vertebra, degenerative disc disease as well as the other conditions that cause chronic back pain.

The devices and methods of the present application can be also be used to treat sciatica. In general, sciatica refers to pain associated with the sciatic nerve which runs from the lower part of the spinal cord (the lumbar region), down the back of the leg and to the foot. Sciatica generally begins with a herniated disc. The herniated disc itself leads to local immune system activation. The herniated disc also may damage the nerve root by pinching or compressing it, leading to additional immune system activation in the area. By implanting the device containing the chemical denervation agent in the intervertebral disc or near a nerve root sciatica can be treated.

### Method of Making Drug Depots

In various embodiments, the drug depot comprising the active ingredients (e.g., the chemical denervation agent, analgesic, anti-inflammatory agent, and/or growth factor) can be made by combining a biocompatible polymer and a therapeutically effective amount of the active ingredients or pharmaceutically acceptable salts thereof and forming the drug depot from the combination.

Where solution processing techniques are used to make the drug depot, a solvent system is typically selected that contains one or more solvent species. The solvent system is generally a good solvent for at least one component of interest, for example, biocompatible polymer and/or therapeutic agent. The particular solvent species that make up the solvent system can also be selected based on other characteristics, including drying rate and surface tension.

Solution processing techniques include solvent casting techniques, spin coating techniques, web coating techniques, solvent spraying techniques, dipping techniques, techniques involving coating via mechanical suspension, including air suspension (e.g., fluidized coating), ink jet techniques and electrostatic techniques. Where appropriate, techniques such as those listed above can be repeated or combined to build up the depot to obtain the desired release rate and desired thickness.

In various embodiments, a solution containing solvent and biocompatible polymer are combined and placed in a mold of the desired size and shape. In this way, polymeric regions, including barrier layers, lubricious layers, and so forth can be formed. If desired, the solution can further comprises, one or more of the following: other therapeutic agent(s) and other optional additives such as radiographic agent(s), etc. in dissolved or dispersed form. This results in a polymeric matrix region containing these species after solvent removal. In other embodiments, a solution containing solvent with dissolved or dispersed therapeutic agent is applied to a pre-existing polymeric region, which can be formed using a variety of techniques including solution processing and thermoplastic processing techniques, whereupon the therapeutic agent is imbibed into the polymeric region.

Thermoplastic processing techniques for forming the depot or portions thereof include molding techniques (for example, injection molding, rotational molding, and so forth), extrusion techniques (for example, extrusion, co-extrusion, multi-layer extrusion, and so forth) and casting.

In other embodiments, biodegradable polymer(s) and one or more therapeutic agents are premixed using non-thermoplastic techniques. For example, the biocompatible polymer can be dissolved in a solvent system containing one or more solvent species. Any desired agents (for example, a radio-opacifying agent, a therapeutic agent, or both radio-opacifying agent and therapeutic agent) can also be dissolved or dispersed in the solvents system. Solvent is then removed from the resulting solution/dispersion, forming a solid material. The resulting solid material can then be granulated for further thermoplastic processing (for example, extrusion) if desired.

As another example, the therapeutic agent can be dissolved or dispersed in a solvent system, which is then applied to a pre-existing drug depot (the pre-existing drug depot can be formed using a variety of techniques including solution and thermoplastic processing techniques, and it can comprise a variety of additives including a radio-opacifying agent and/or viscosity enhancing agent), whereupon the therapeutic agent is imbibed on or in the drug depot. As above, the resulting solid material can then be granulated for further processing, if desired.

Typically, an extrusion processes may be used to form the drug depot comprising a biocompatible polymer(s), therapeutic agent(s) and radio-opacifying agent(s). Co-extrusion may also be employed, which is a shaping process that can be used to produce a drug depot comprising the same or different layers or regions (for example, a structure comprising one or more polymeric matrix layers or regions that have permeability to fluids to allow immediate and/or sustained drug release). Multi-region depots can also be formed by other processing and shaping techniques such as co-injection or sequential injection molding technology.

In various embodiments, the depot that may emerge from the thermoplastic processing (e.g., pellet, strip, etc.) is cooled. Examples of cooling processes include air cooling and/or immersion in a cooling bath. In some embodiments, a water bath is used to cool the extruded depot. However, where water-soluble therapeutic agents are used, the immersion time should be held to a minimum to avoid unnecessary loss of therapeutic agent into the bath.

It will be apparent to those skilled in the art that various modifications and variations can be made to various embodiments described herein without departing from the spirit or scope of the teachings herein. Thus, it is intended that various embodiments cover other modifications and variations of various embodiments within the scope of the present teachings.

What is claimed is:

1. A device for treating chronic back pain in a patient in need of such treatment, the device configured to deliver a chemical denervation agent to an effective treatment zone comprising a nerve at a target region of the spine so as to chemically ablate the nerve.

2. A device according to claim 1, wherein the device is a catheter configured for positioning in the effective treatment zone of a vertebrae wherein the effective treatment zone comprises the basivertebral nerve.

3. A device according to claim 2, wherein the vertebrae is in the lumbar region of the spine.

4. A device according to claim 1, wherein the device is an implantable biodegradable drug depot that releases at least one chemical denervation agent in the effective treatment zone when implanted in or adjacent to the effective treatment zone so as to chemically ablate the nerve over time as the chemical denervation agent is released.

5. A device according to claim 4, wherein the device is configured to be implanted into the effective treatment zone and the effective treatment zone comprises a basivertebral nerve.

6. A device according to claim 5, wherein the vertebrae is in the lumbar region of the spine.

7. A device of claim 1, wherein the implantable biodegradable drug depot comprises an immediate release component that releases the chemical denervation agent within 24 hours and a sustained release component that releases the chemical denervation agent over a period of at least 1 to 3 days after implantation.

8. A device according to claim 2, wherein the drug depot comprises a polymer and the polymer comprises about 60% to 99% of the total weight % of the drug depot and the effective treatment zone comprises the basivertebral nerve.

9. A device according to claim 4, wherein the device is a drug depot that releases (i) a bolus dose of the chemical denervation agent within the effective treatment zone over a period of up to 3 days and (ii) a sustained release dose of the chemical denervation agent within the effective treatment zone over a period of up to 3 months.

10. A device according to claim 4, wherein the device releases about 20% to about 99% of the chemical denervation agent relative to a total amount of the chemical denervation agent loaded in the device over a period of about 3 days to about 1 month after the device is administered within the effective treatment zone.

11. A device according to claim 1, wherein the chemical denervation agent comprises neurolytic agents, hyaluronidase, ethanol, ethyl alcohol, neurotoxin agents, magnetic particles containing neurotoxins, Botox b phenol, hypertonic saline, and combinations thereof.

12. A device according to claim 4, wherein the drug depot further comprises a growth factor, an analgesic, an anti-inflammatory agent or a combination thereof and the drug depot comprises at least one biodegradable polymer comprising one or more of poly(lactide-co-glycolide) (PLGA), polylactide (PLA), polyglycolide (PGA), D-lactide, D,L-lactide, L-lactide, D,L-lactide-co-ε-caprolactone, D,L-lactide-co-glycolide-co-ε-caprolactone or a combination thereof.

13. A device according to claim 7, wherein the growth factor is disposed in one or more components that are separate from the immediate release component and the sustained release component of the drug depot.

14. A device according to claim 1, wherein the drug depot is polymerizable in-situ or curable in-situ in the effective treatment zone comprising a nerve.

15. A device for treating chronic back pain in a patient in need of such treatment, the device being implantable in or near an the basivertebral nerve of the patient, the device comprising a chemical denervation agent and having an immediate release component configured to release an effective amount of the chemical denervation agent within 24 hours to ablate at least a portion of the basivertebral nerve of the patient.

16. A device according to claim 15 further comprising a sustained release component contacting the immediate release component to provide sustained release of the chemical denervation agent over a period of up to 1 year to ablate the basivertebral nerve so as to treat chronic back pain of the patient.

17. A method for treating chronic back pain in a patient in need of such treatment, the method comprising the steps of positioning a delivery device for delivery of a chemical denervation agent in or near an effective treatment zone containing a basivertebral nerve for administering a chemical denervation agent to chemically ablate at least a portion of the basivertebral nerve so as to treat chronic back pain of the patient.

18. A method according to claim 17, wherein the delivery device is positioned in or near the effective treatment zone by inserting a needle or cannula into the vertebrae such that the inserted end of the needle or cannula is inside the vertebrae in or near the effective treatment zone; and delivering the chemical denervation agent to the effective treatment zone; and removing the needle or cannula from the vertebra.

19. A method according to claim 16, wherein the chemical denervation agent is in a biodegradable drug depot comprising a polymer and the chemical denervation agent to chemically ablate at least a portion of the basivertebral nerve, the drug depot having an immediate release component configured to release an effective amount of the chemical denervation agent within 24 hours and a sustained release component to provide sustained release of the chemical denervation agent over a period of up to 3 months to treat chronic back pain of the patient.

20. A method according to claim 19, wherein the drug depot further comprises a growth factor, an analgesic, an anti-inflammatory agent or a combination thereof.
